(19) United States
(12) Patent Application Publication
(10) Pub. No.: US 2017/0354367 A1
(43) Pub. Date: Dec. 14, 2017

(54) PROSTHETIC DISTAL FORCE MEASUREMENT DEVICE

(71) Applicant: ALPS SOUTH INTELLECTUAL PROPERTY MANAGEMENT, LLC, ST. PETERSBURG, FL (US)

(72) Inventors: Aldo A. Laghi, Clearwater, FL (US); Eric Prast, St. Petersburg, FL (US); Kevin McLoone, Dunedin, FL (US)

(73) Assignee: ALPS SOUTH INTELLECTUAL PROPERTY MANAGEMENT, LLC, ST. PETERSBURG, FL (US)

(21) Appl. No.: 15/380,400

(22) Filed: Dec. 15, 2016

Related U.S. Application Data
(63) Continuation-in-part of application No. 14/172,123, filed on Feb. 4, 2014, now abandoned.

(51) Int. Cl. A61B 5/00; A61F 2/78; A61F 2/80; A61F 2/76; A61F 2/70

(52) U.S. Cl. CPC A61B 5/4851; A61B 5/6811; A61F 2/7812; A61F 2/80; A61F 2002/705; A61F 2002/704; A61F 2002/7635

(57) ABSTRACT

The following invention is a device for measuring the force applied by the residual limb of an amputee to the distal region of a socket. The device has an upper surface with a “force sensor measuring region” that maintains a central location on the upper surface of the DFMD. The DFMD is affixed to the inside surface of the distal most area of the socket and maintains a permanent location of the “force sensor measuring region” of the device. Regardless of physical characteristics or changes to the socket, liner, socks, proper or improper placement of the limb into the socket, the consistent location of the “force sensor measuring region” on the DFMD provides congruent force data as it relates to the force applied by the socket to the distal area of the residual limb. The data collected by the DFMD is processed and modified by a software algorithm into meaningful data for the user and/or medical professional. Applicable uses for the data relate to the fit of the socket, limb volume management strategy, and vacuum suspension efficacy.

### CROSS-REFERENCE TO RELATED APPLICATION

This application is a continuation-in-part application of application entitled “Prosthetic Distal Force Measurement Device”, Ser. No. 14/172,123 filed Feb. 4, 2014, the disclosure of which is hereby incorporated by reference herein.

### BACKGROUND OF THE INVENTION

### Field of the Invention

This document generally relates to the field of prosthetic liners and sockets having information systems for managing the comfort level of the user.

### Description of the Background Art

A lower limb prosthetic liner provides optimum functionality when the distribution of patient's weight in a prosthetic socket is one third distal and two thirds proximal. Currently there is no feasible way for a prosthetist to measure weight distributing during the fitting of a new socket to an amputee. The prosthetist relies on experience to make an approximate evaluation of weight distribution. An inappropriate distribution of weight may result in discomfort poor gait, and damage to the limb. Many of the lower limb amputees are vascular with poor circulation in the residual limb and poor sensory perception. Excessive pressure points are likely to go unnoticed by the amputees which result in wounds that are difficult to heal. Even if the initial socket is properly made and allows an optimal distribution of weight therein, with the maturing of the residual limb over time, the size of the limb shrinks and the pressure of the distal end of the residual limb against the distal end surface of the socket increases. Vascular patients are subjected to positive and negative volume fluctuations more than healthy individuals and such fluctuations affect the distribution of weight in the socket. This problem is address by the prosthetist by training the patient to use additional prosthetic socks or a different ply sock for the management of the volume fluctuations of the residual limb. This solution is only partially effective with many older amputees who are not able to assess how many prosthetic socks or what ply of sock to use.

As a solution to this problem, prosthetic socks and/or liners now on the market have been designed to have residual limb monitoring systems built into them. For example, Patent Application No. US 2012/0226197 to Sanders, et al. disclose a prosthetic liner having sensors built into the liner for monitoring various activities of the amputee that effect the volume of an amputee's residual limb. The sensors on the liner further include a transmitter for transmitting the collected data to a remote computer system of the user, a doctor, or prosthetist for developing a sock monitoring strategy. Such data is useful in determining what adjustments must be made to the interface between the residual limb and socket, such as adding or eliminating extra socks, or using socks of a different ply thickness.

However, to date, such prior art systems either have the sensors built into the liner or into the socket. Such liners and sockets are complicated to manufacture and expensive, not to mention the fact that costs rise dramatically if they have to be replaced. Thus, the present invention provides another and simpler solution to this problem without having to mount the sensors in the liner and/or socket. The present invention provides a simple resilient insert having force sensing and transmitting electronics for continuous and accurate measurement of the distal contact of an amputee's residual limb against the inside distal surface of a prosthetic socket.

The field of prosthetics as it relates to lower limb amputees generally relies on the craftsmanship and skill of an individual, often CPO or technician, to fabricate the socket. Once the socket is fabricated the means of measuring the fit is often overlooked due to the complexity of taking such a measurement. The typical residual lower limb to socket configuration is comprised of a lower limb, donned over said lower limb a prosthetic liner, said lower limb with donned prosthetic liner then into a socket. Prosthetic liners come in several variations including thermoplastic elastomer (TPE), urethane, and silicone. Liners comprised of these material variations often have an outer fabric covering. Sockets are generally fabricated using composite materials such as fiberglass or carbon fiber though a check socket formed using a thermoplastic maybe used on a temporary basis.

For the purpose of discussion, the method of determining the fit of the residual lower limb to socket will be compared to the fit of a shoe over a non-ambulatory individual. Said fit is often determined by the feel during walking, standing, and by pressing on the soft shoe material at the end of the toe to determine if the sizing is appropriate. For adults this process is often intuitive and obvious as foot growth is no longer a factor and the individual adult has already formed an idea on how the correct shoe should “feel.” Certainly the “feel” portion is non-quantitative and based on experience. However, measurements of the foot prior to the shoe selection can often result in the correctly sized shoe, or at least very close. The average individual is able to appropriately and satisfactorily determine their own shoe fit using the described method. One within the field of prosthetics can appreciate the incongruences between the described method of determining shoe fit and the methods in determining socket fit.

Notably, in the time period between the initial amputation and receipt of the first socket/prosthesis assembly, the residual limb experiences drastic volume change. The tissue towards the bottom of the limb is where the majority of the shrinkage occurs. While a shoe is generally soft and pliable, and in some cases open, the socket is rigid and opaque. This makes it impossible to observe, at least from an external perspective, if areas exist within the socket that does not contact the residual limb. Regions of the residual limb with excessive pressure cannot be observed externally from the socket.

In general terms the residual limb and corresponding socket can be considered conical in which the proximal cross-sectional area is greater than the distal cross-sectional area. The result of this tapered geometry is that the forces are not only applied distally but are also applied against the walls of the socket. During socket construction a prosthetic liner is usually considered to be a component of the assembly. In many instances this is similar to wearing a sock over a foot during the show trial process. As a result, the inside surface of the socket is offset some predetermined distance from the outside surface of the residual limb to account for said prosthetic liner. A key difference is that said prosthetic liner is 1 to 9 mm in thickness. In such an assembly, the prosthetic liner functions to reduce the shear forces that would be present if the residual limb were in direct contact with the inside surface of the socket. Regardless of composition, prosthetic liners utilize materials that with elasticity to provide even distribution of forces between the residual limb and socket. This assists in reducing the effects of any socket defects during construction and also accounts for slight volume changes in the residual limb. The prosthetic liner also provides cushioning at the regions of the residual limb that that have experienced the trauma of an amputation or have paid due the nerve endings.

Another dissimilarity in the foot to residual limb comparison is in regard to the sensitivity of nerve endings within the residual limb. The sensations experienced when wearing a socket are new and therefore make it difficult for the amputee to determine whether the socket “feels” good. Due to the new environment the amputee is able to decipher pain in the residual limb but often such feelings may not be necessarily due to an improper fit. It may be as a result of the amputation. Conversely, an experienced amputee who's residual limb has adapted to the pressure environment of a socket may not be able to detect, when fitted with a new socket, if there are regions that do not have contact with the socket. This could result in pistoning or other adverse effects on the residual limb. Considering that the vast majority of amputees have a compromised vascular system it is important that the socket fit is appropriate to facilitate the health of the residual limb and thus the patient. In an ideal scenario, the inside surface of the socket and the outside surface of the prosthetic liner are flush, or a perfect mate when the socket is donned.

Various inventions have been devised to measure the fit of the residual limb into the socket. U.S. Pat. No. 8,784,340, describes a prosthetic sock monitoring system with one or more sensors coupled to a prosthetic sock. While providing a possible solution, said sock monitoring system does not provide a solution to the powering of the sensors. Attaching wires or batteries to said sensors would not be feasible as the prosthetic sock is a laundered article. Furthermore, the coupling of the sensor and accompanying means of power would create a pressure point between the residual limb and rigid socket wall resulting in discomfort and a possibly harm to the residual limb. Lastly, and most prominently, the meaningfulness of the data gathered from any sensor is based on the stability of the location in which it is originally installed. Due their construction and material properties prosthetic socks stretch considerably. Over the course of laundering cycles the prosthetic sock changes size. Such an invention would require alignment marks corresponding to marking on the residual limb or prosthetic liner to ensure that the sensor location is in the same location from one donning to the next. The stretch of said sock further diminishes the likelihood that the amputee is able to repeatedly don said sock. In the unlikely event that all such circumstances are overcome and amputee is able to insert said sock into the socket without shift, it would be an increasingly difficult challenge should the system require donning of additional socks to compensate for volume loss. Then, the prosthetic assembly would have to be removed, the procedure repeated with an additional prosthetic sock over said sensor coupled sock, without shift, and the assembly reinserted into the socket.

Another method for determining proper socket fit is by incorporating strain gauges in various locations on the socket wall. Such a method requires extensive skill to fabricate but also extensive testing to determine how the measured results relate to the fit of the residual limb within the socket.

Another method for determining proper socket fit is by inserting a soft and pliable material into the distal region of the socket, inserting the limb, performing an activity involving the limb such as walking, removing the socket, and evaluating the shape of the inserted soft and pliable material. Said soft and pliable material is non-compressible and has nowhere to escape when confined between surfaces. As a result, the soft and pliable material prevents the residual limb and liner assembly from full contact, often misrepresenting a void.

### SUMMARY OF THE INVENTION

The present invention relates generally to a pressure measurement device that is inserted into a prosthetic socket at the distal end and located between the socket and a socket liner. The device comprises a molded replaceable cushion insert made of resilient silicone or polymeric material shaped to have a lower surface to complementally fit firmly against the interior distal end surface of the socket and an upper concave surface that is contiguous with the concave surface of the distal interior of the socket. The insert is removable from the socket and therefore replaceable. The insert further includes at least one force sensor and electronic receiving and transmitting circuitry associated therewith either embedded or removable mounted therein. In the embodiment where the force sensor and circuitry are embedded therein such as during the molding process, the entire device would be replaceable. In the embodiment where the force sensor and circuitry are removably mounted therein, only the force sensor and/or the transmitting circuitry would have to be replaced. Each force sensor is disposed in the insert and adapted to detect downward pressure caused by a distal end of an amputee's residual limb against the distal end of the socket. Throughout the day, an amputee's residual limb may change in volume such as by swelling or contraction of the residual limb for various reasons. Such volumetric changes will affect the fit of the residual limb within the socket which in turn affects the downward pressure of the residual limb against the distal end of the socket. The present invention is designed to measure these pressure variations and provide feedback to the user, rehabilitation doctor, and/or CPO (Certified Prosthetist/Orthotist). The electronic circuitry transmits the pressure measurements wirelessly to a personal computer or mobile computing device throughout the day. This invention is designed to systematically inform the user, rehabilitation doctor, and/or a CPO (Certified Prosthetist/Orthotist) of the degree of force or pressure that the distal end of the residual limb is exerting against the distal end of the prosthetic socket throughout the time of use. Accordingly, such information is useful in determining whether a new socket, a new sock, additional socks or elimination of socks may be needed.

In a first embodiment, the insert is molded of a resilient silicone or polymeric material to have a concave shape with the concave side molded to match the interior concave surface of a distal end of a socket. This embodiment could be molded to have at least one force sensor and transmitter circuitry embedded therein or molded to have a cavity in the concave side in which the at least one force sensor and transmitting circuitry can be removably secured.

In a second embodiment, the insert is also molded to have a concave shape with the concave side molded to match the interior concave surface of a distal end of a socket. However, in this embodiment, the concave side of the insert includes an extension leading therefrom which is complementally shaped to fit inside a well or recess defined by a lower section of the socket depending from the distal end thereof. This embodiment could be molded to have at least one force sensor and transmitter circuitry embedded in the extension or molded to have a cavity in the extension in which the at least one force sensor and transmitting circuitry can be removably secured.

The present invention is a device for measuring the force applied by the residual limb distal region of an amputee to the distal region of a socket. Accordingly, the Distal Force Measuring Device (hereinafter, “DFMD”) is an enclosed body with a concave upper surface that is affixed to the inside surface of the socket lying on an axis approximately central to the socket, albeit the socket is asymmetrical it will be considered conical for discussion of said axis. A fundamental concept of the present invention is that the force between the distal region of a residual limb and socket will fluctuate between a series of values representing healthy and unhealthy conditions. During periods of rest and activity, the DFMD measures the distal force between socket and residual limb. In one embodiment the DFMD monitors the force readings, executes algorithms, and provides feedback to the patient concerning the fit of the socket. In another embodiment, the DFMD transmits the force readings to a separate cloud based database.

An object of the present invention is to utilize the distal force data to create a limb-to-socket force tolerance (hereinafter “force tolerance”) from which a range of force values may be derived to suggest acceptable distal force conditions. Likewise, limb-to-socket force values that are outside of the force tolerance are used to represent unacceptable distal force conditions. The data collected by the DFMD enables the user and/or medical professional to understand said residual limb conditions and make adjustments to prosthesis to ensure the health of the residual limb.

A method of the present invention in making said adjustments to said prosthesis is to provide instructions for volume management of the limb within the socket. If the force measured by the DFMD is outside of the force tolerance for an excessive time period the patient is automatically prompted to take action. More specifically, if the force is in excess of the force tolerance the patient is prompted to reduce the number of sock plys. If the force is below the force tolerance the patient is prompted to increase the number of sock plys.

In a second object of the present invention, the force readings relate to the proper or improper sizing of the socket with respect to the limb. It is well known that the residual limb experiences drastic volume reduction and shape within the initial time period following the amputation of said limb. As the reduction in residual limb volume occurs, the net result may be measured by the distal force against the DFMD. If the socket is too large, the distal force reading would be excessively high implying that the force value is beyond the threshold that is correctable by the addition of sock plys.

In a third object of the present invention, the force readings relate to the measurement of vacuum within the socket. The applicability of this embodiment is unique in prosthesis' utilizing vacuum suspension systems. The efficacy of the vacuum system is based on the vacuum level being properly maintained. Should a failure occur in the system, and a leak develop, the distal force detected by the DFMD would detect a shift in force data as compared to previous average values.

The present invention is a distal force measuring device with a sensor that is affixed to the distal inside surface of the socket allowing for measurements to be repeatable and consistent. The DFMD measures values that relate specifically to the residual limb, socket, liner, and prosthetic sock(s).

### BRIEF DESCRIPTION OF THE DRAWINGS

For a more complete understanding of the present disclosure and its advantages, reference is now made to the following descriptions, taken in conjunction with the accompanying drawings, in which:

FIG. 1 shows a cross-sectional view of the first embodiment of the present invention inserted between a prosthetic liner and the distal end of a prosthetic socket with the electronic components embedded within the insert.

FIG. 2 shows a cross-sectional view of the first embodiment of the present invention inserted between a prosthetic liner and the distal end of a prosthetic socket modified to have a central opening for receiving a locking pin of a locking liner.

FIG. 3 is a cross-sectional view of a second embodiment of the present invention inserted between a prosthetic liner and the distal end of a prosthetic socket and having a housing for the receiver/transmitter circuitry separate from the force sensor.

FIG. 4 is a cross-sectional view of the second embodiment of the present invention inserted between a prosthetic liner and the distal end of a prosthetic socket modified to have a central opening for received a locking pin of a locking liner.

FIG. 5 is a cross-sectional view of a third embodiment of the present invention inserted between a prosthetic liner and the distal end of a prosthetic socket and having a housing for the receiver/transmitter circuitry separate from the force sensor.

FIG. 5a is an enlarged cross-sectional view of the encircled structure illustrated in FIG. 5 illustrating a first modification of the force sensor and housing for the receiver/transmitter circuitry separate from the force sensor.

FIG. 5b is an enlarged cross-sectional view of the encircled structure illustrated in FIG. 5 illustrating a second modification of the force sensor housing for the receiver/transmitter circuitry separate from the force sensor.

FIG. 6 is a cross-sectional view of the third embodiment of the present invention inserted between a prosthetic liner and the distal end of a prosthetic socket modified to have a central opening for receiving a locking pin of a locking liner.

FIG. 7 is a cross-sectional view of the third embodiment shown in FIG. 3 along the lines VII-VII.

FIG. 8 is an illustrated view of how the present invention communicates with computer systems via Bluetooth technology.

FIG. 9 is an exploded view of an individual with a residual limb, the liner, the sock, and the prosthetic.

FIG. 10 is a cross-sectional view of the present embodiment of the invention showing the force sensor's placement within the prosthetic.

FIG. 11 is a cross-sectional view of an alternate embodiment of the present invention where the force sensor is split into a plurality of components.

FIG. 12 is a side cross-sectional view of the force sensor.

FIG. 13 is an isometric view of the force sensor shown in FIG. 12.

FIG. 14 is an isometric view of the force sensor component of FIG. 11.

FIG. 15 is a side cross-sectional view of the force sensor of FIG. 14.

Similar reference numerals refer to similar parts throughout the several views of the drawings.

### DETAILED DESCRIPTION OF THE DRAWINGS

The various components of the present invention, and the manner in which they interrelate, are described in greater detail hereinafter.

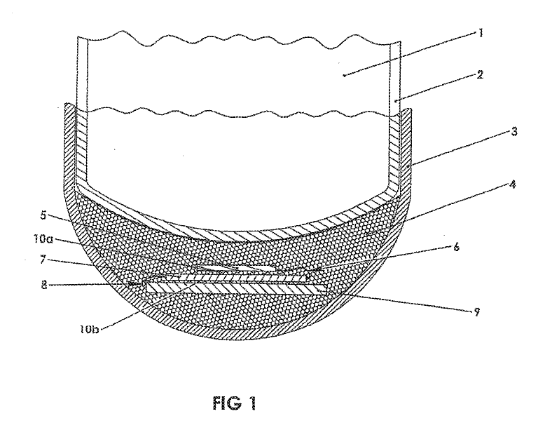

Referring to FIG. 1, a residual limb of an amputee is denoted by the numeral 1. As illustrated, prosthetic liner 2 is donned onto the residual limb and inserted into prosthetic socket 3. A first embodiment of the present invention includes an insert 4 comprising an elastomeric member adapted to fit between the distal end of the liner and the distal interior of the socket. Embedded within the elastomeric member 4 is force sensor 5 electrically connected to data collecting and transmitting circuitry 7 via conductors 6, and battery 9 electrically connected to circuitry 7 via conductors 8. The insert 4 is removable and therefore replaceable should failure of the force sensor or any electronic component be necessary, or simply because the elastomeric member itself has reached its fatigue state.

The present invention's insert 4 is not limited for use with any particular prosthetic liner and socket arrangement. The embodiment of FIG. 1 could be employed with any conventional suction and/or suspension type of prostheses as, for example, those disclosed in U.S. Pat. Nos. 6,544,292; 5,314,497; 5,571,208; 4,479,272; or 8,523,951, all incorporated herein by reference. Although not shown, the socket of the embodiment of FIG. 1 could be connected to a pylon arrangement for a prosthetic foot as, for example, illustrated in FIG. 7 of U.S. Pat. No. 5,888,217, incorporated herein by reference.

The insert 4 is made of a thermoplastic elastomeric material, preferably gel materials, for example by injection molding techniques, such as silicone, copolymer Styrenic gels, polyurethane, block copolymers or other TPE elastomers. A wide variety of thermoplastic materials that could be used to manufacture the present invention are disclosed in U.S. Pat. No. 5,633,286, incorporated herein by reference.

The force sensor 5 is comprised of an ultra-thin flexible force sensing element used to measure a relative change in force or applied load. It may be used for measuring rate of change and identifying force thresholds to trigger an appropriate action. The sensor may be also used as a means of switching a device on therefore detecting presence, contact, and/or touch. The device used in this design is a durable piezoresistive force sensor created in various shapes and sizes tailored to the gel apparatus. The resistance measured is inversely proportional to the applied force. One type of force sensor is the FlexiForce® sensor manufactured and patented by Tekscan, this type of sensor provides a linear force measurement ±3% and can withstand high temperature environments up to 400° F. (HT201). The FlexiForce® sensor can measure up to 100 lbs or force with <5 microseconds response time. The sensor 5 is connected through pins to a flexible conductive fabric, thread, or elastic bonded wire 6 which carries the signal to a receiver/transmitter microcontroller 7. The force sensor cables may also contain a resistor or resistive device to provide a ground reference to the controller.

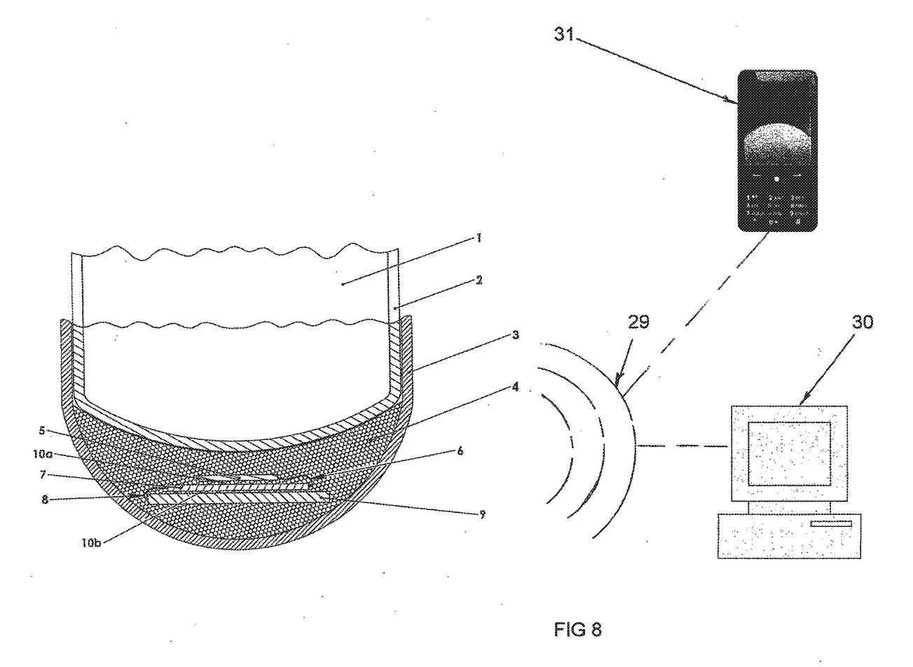

The receiver/transmitter microcontroller 7 receives data from the force sensor 5 and relays such data via Bluetooth technology 29 to an external electronic device such as a personal computer 30 or cell phone 31 as illustrated in FIG. 8. The receiver/transmitter microcontroller 7 may include an RN41 which is a small form factor, low power: class 1 Bluetooth module that operates on the 2.4 GHz ISM band with a maximum distance of 100 M. The RN41 delivers up to 3 Mbps data rate and uses FHSS/GFSK modulation with 79 channels at 1 MHz intervals. The modules uses a 128 bit encryption key for secure connections and uses a UART local over the air RF-configuration. This device has auto-discovery/pairing without software configuration. It provides an auto-connector master, IO pin (DTR line) and character based trigger modes. Although one type of receiver/transmitter is described, the present invention is not so limited and will operate with any other conventional receiver/transmitter microcontroller.

One of the main advantages of this RF module and other types are the ultra-low power sleep mode that provides efficient battery use while asleep. The module will maintain a heartbeat looking for a control signal to wake up and transmit data again. The receiving data may be processed, saved, cataloged, and displayed to the end user. The EEDS may be a hand-held electronic device or software application, the software is designed to be compatible with Android, iOS, or other major smart-phone device operating systems. The software application is an integral component of the system. The software is necessary in order to record a historical trend of the patients fit as well as perform sensor calibration. The EEDS can be also used by the clinician to provide feedback on the socket fit. The software also allows the ability to set how many data-points per day, and generate a report and send it through email. The EEDs also has the ability to alert the patient of low battery levels in the device.

The battery 9 is a power supply of the PSBL type and may be lithium ion, lithium polymer, lithium iron phosphate, nickel-cadmium, or any rechargeable battery source. A single supply is used with an on-board voltage regulator to power 1.8V, 3.3V, and 5V levels. The cells may be configured in a single or multiple parallel, series, or similar layout.

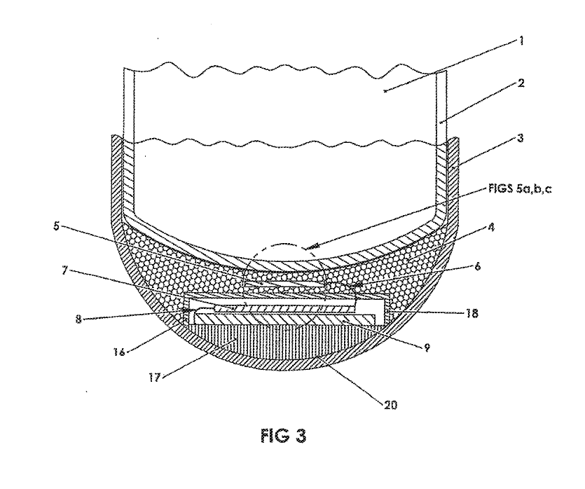

As a wearable device, battery life, energy density, accessibility, and rechargeable capabilities are essential. In the first embodiment, the force sensor, electronic circuitry and batter are adhered to each by adhesive layers 10a and 10b and full embedded within the elastomeric material 4 during the molding process. Thus, the entire unit may be disposable or, if not, may include a conventional recharging circuit (not shown) by plugging in the apparatus through a charging port. In the second and third embodiments illustrated in FIGS. 3-6, the battery is removable and can either be replaced or recharged using any conventional recharging circuit.

All of the embodiments disclosed herein could be molded in various sizes to fit different size sockets or could be custom fitted to the distal interior surface of a prosthetic socket that may be in use by the user.

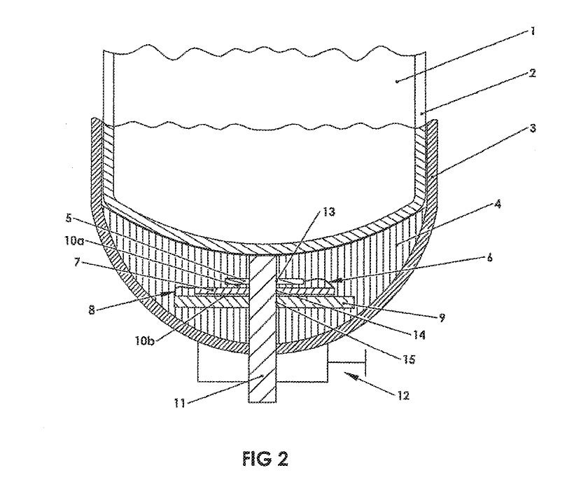

Referring to FIG. 2, a modification to the embodiment shown in FIG. 1 is illustrated. Liner 2 may have a locking pin 11 attached to the distal end thereof. Each of the sensor 5, circuitry 7, and battery 9 include a central opening 13, 14, and 15, respectively, for permitting a locking pin 11 to pass therethrough and be locked in place by a locking mechanism 12. Although the specifics of the locking arrangement is not shown, any conventional locking liner arrangement, such as that disclosed in U.S. Pat. No. 8,394,150, could be modified to include the present invention.

Referring to FIG. 3, a second embodiment of the invention is illustrated. This embodiment is similar to the embodiment of FIG. 1, with the exception of including a housing 16 for the circuitry 7 and battery 9. The housing 16 could be molded together with the insert 4, or the insert 4 could be molded to have a cavity 18 for receiving the housing 16. Whether the housing is molded with the insert, or fitted into the cavity, the bottom edge of the housing sidewall are disposed to rest against the interior surface of the distal end of the socket. This embodiment further includes cap 17 adapted to fit into the lower end of the housing and has a lower surface 20 complementally configured to the distal interior surface of the socket to provide support for the circuitry and battery in the housing. Thus, in this embodiment, the circuitry 7 and battery 9 can be removed from the housing for servicing, or the housing with the circuitry and battery therein can be removed from the insert 4 for servicing.

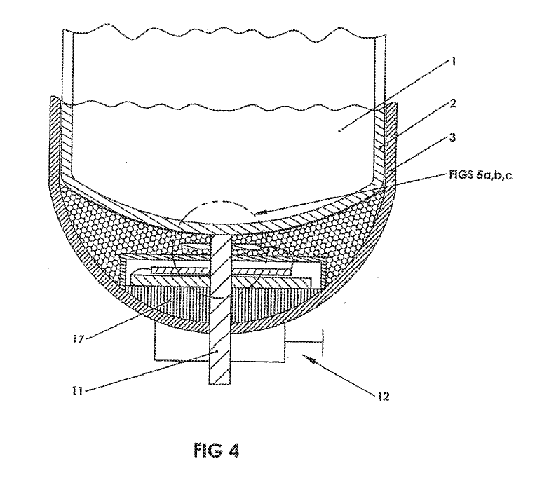

Referring to FIG. 4, a modification to the embodiment shown in FIG. 3 is illustrated. As in the embodiment of FIG. 2, liner 2 may have a locking pin 11 attached to the distal end thereof. Each of the sensor 5, circuitry 7, and battery 9 include a central opening 13, 14, and 15, respectively, for permitting a locking pin 11 to pass therethrough and be locked in place by a locking mechanism 12.

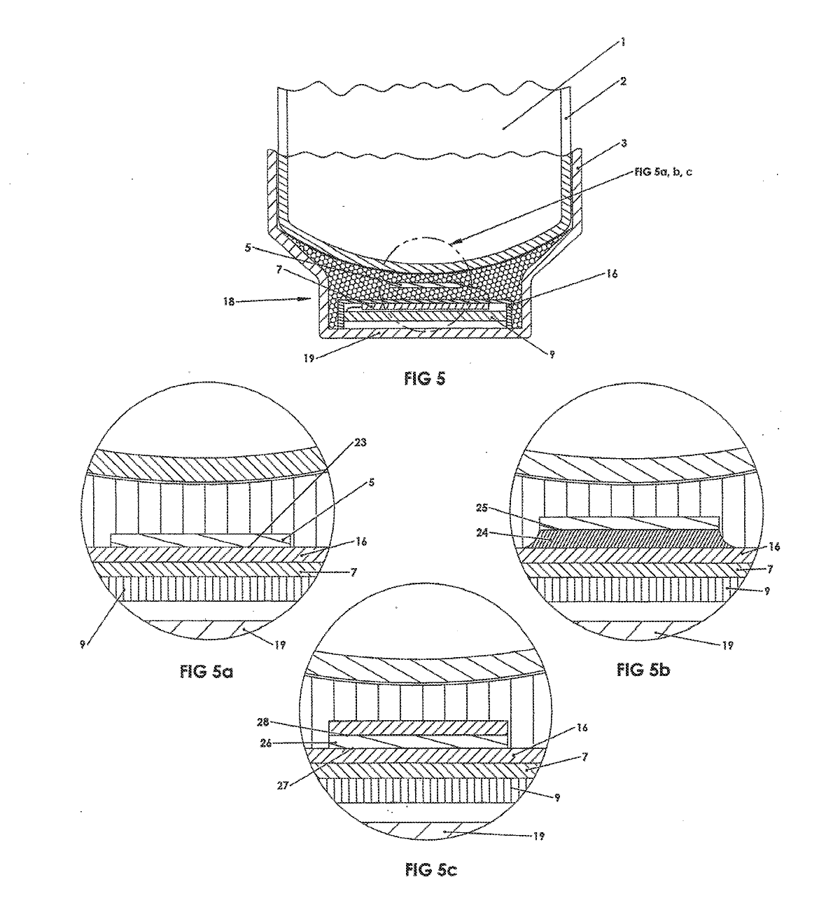

Referring to FIG. 5, a third embodiment of the present invention is illustrated. This embodiment is similar to the embodiment of FIG. 3 with the exception of including a well or recess 18 extending from the lower end of the socket. The well or recess 18 could have any cross-sectional shape, such as square or circular. The well or recess terminates in a flat bottom surface 19 on which the lower edge of housing 16 is supported. Therefore, this embodiment does not require a cap as in the embodiment of FIG. 3. All the other features of this embodiment are the same as in the embodiment of FIG. 3 and thus, will not be repeated here.

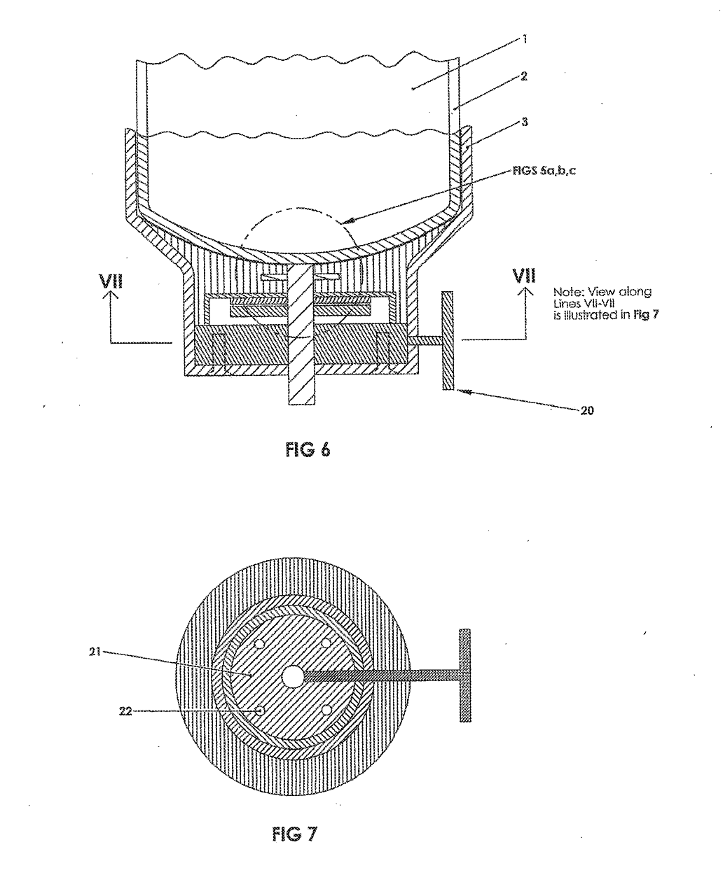

Referring to FIG. 6, a modification to the embodiment shown in FIG. 5 is illustrated. As in the embodiments of FIGS. 2 and 3, liner 2 may have a locking pin 11 attached to the distal end thereof. Each of the sensor 5, circuitry 7, and battery 9 include a central opening 13, 14, and 15, respectively, for permitting a locking pin 11 to pass therethrough and be locked in place by a conventional locking mechanism 20 such as that disclosed in U.S. Pat. No. 8,444,702. It is noted that in this embodiment, the locking mechanism 20 is mounted within the socket whereas the locking mechanism of the embodiments of FIGS. 2 and 4 are mounted exterior to the socket.

In FIG. 7, a cross-sectional view of the cross-section through lines VII-VII in FIG. 6 is illustrated. The locking mechanism may include a plate 21 having screw holes 22 for fasteners such as screws (not shown) for securing the locking mechanism and also may be used to attach a pedestal (not shown) for attaching a pylon and an artificial foot prosthesis (not shown).

Referring to FIGS. 5a, b, and c, three possible modifications of the embodiments of 3, 4, 5, and 6 are illustrated. The force sensor 5 in the embodiments of FIGS. 3, 4, 5, and 6 is shown detached from the housing 16 and is embedded within the insert 4. The force sensor 5 is electrically connected to circuitry 7 via conductor 6. Thus, the conductor 6 must extend within the housing sufficiently in order to remove the circuitry 7 and battery 9 from the housing 16 for servicing. On the other hand, should the housing with circuitry and batter therein be removed from the cavity 18 in the insert 4, the conductor 6 must be disconnected from the circuitry in order to service the circuitry and the battery. In all three modifications shown in FIGS. 5a, b, and c, the force sensor 5 is attached to the housing 16. In FIG. 5a, the force sensor is attached directly to the top surface of housing 16 by adhesive layer 23. In FIG. 5b, the top surface of the housing is formed with a boss 24 to which sensor 5 is attached by an adhesive layer 25. In FIG. 5c, a separate plastic sheet 26 is mounted to the top surface of the housing by an adhesive layer 27 to which the sensor 5 is attached by an adhesive layer 28. The boss 24 in FIG. 5b and plastic piece 26 in FIG. 5c provide extra backing for the sensor 5 and also dispose the sensor 5 more approximate to the bottom of the liner 2 rendering it more sensitive to downward pressure from the residual limb.

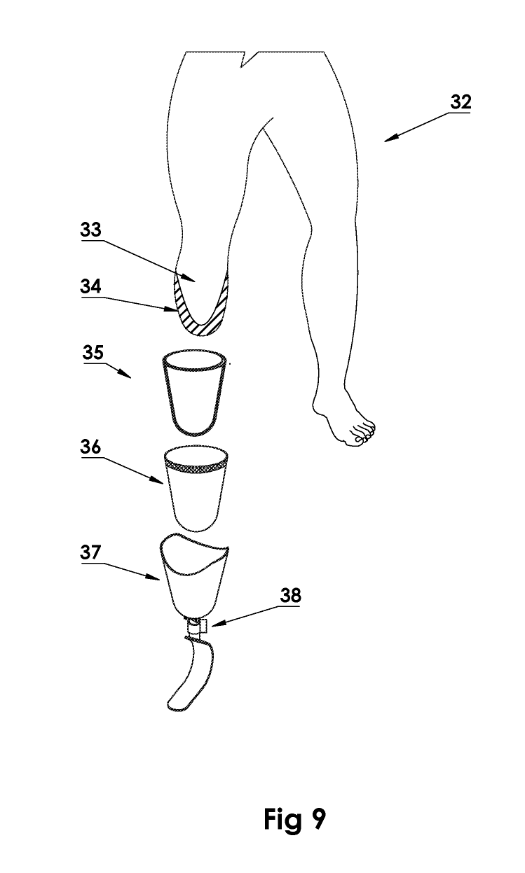

Illustrated in FIG. 9, an exploded view of the present invention in conjunction with other elements that are common with a below the knee amputee. The lower torso 32 of the below knee amputee is shown along with the residual limb 33. A shaded region is shown illustrating the region of limb volume fluctuation 34. The first element that interfaces with the residual limb is the liner 35. In most instances the liner is comprised of a thermoplastic elastomer that contacts the skin of the residual limb. The outer surface of the thermoplastic elastomer liner (hereinafter “liner”) is preferably comprised of a knitted fabric construction. The illustrated liner is considered a suction or suspension liner which means that the prosthesis relies on vacuum to suspend the prosthesis onto the residual limb. In many instances a sleeve (not illustrated) would go over the socket and liner to provide an additional seal to ensure proper vacuum.

The next component of an assembly common to the present invention is a sock 36 or prosthetic sock. These are common within the market place and typically are of knit construction using synthetic fibers. Common thicknesses available for prosthetic socks are 1, 3, and 5 ply. Due to the knit construction, and to facilitate donning and doffing, prosthetic socks are capable of stretch in the vertical and horizontal direction in excess of 40 percent. Illustrated is a singular prosthetic sock but it should be noted that an assembly utilizing the present invention may be comprised of a plurality of prosthetic socks that may differ in sock plys. The prosthetic sock is donned over the outside surface of the liner such that the inside surface of the sock interfaces with the outside surface of the liner. The outside surface of the sock interfaces with the inside surface of the socket. It should be noted that in an assembly comprising a plurality of socks the outermost sock would have an outer surface in contact with the socket. Said socket then attached to various mechanical devices including a prosthetic foot. The socket, prosthetic foot, and all related mechanical components there between represent what is considered the prosthesis 37.

The present invention is a prosthetic distal force measurement device (“DFMD”) 38 for measuring the distal force between the socket and residual limb of an amputee that is affixed to the distal most inside surface of the socket.

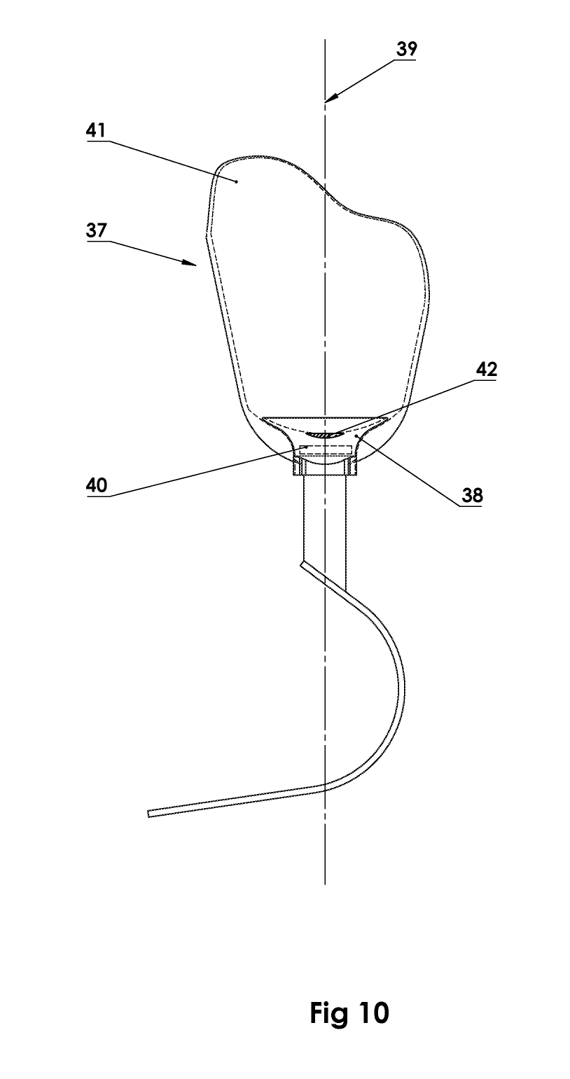

FIG. 10 shows an embodiment of the present invention in which, the DFMD 38 is a single enclosure that houses the sensor and processor 40. The enclosure 40 is affixed to the inside surface 41 of the socket at the lowest point of the socket axis 39. (Sockets are generally asymmetrical as they take the form of the residual limb of an amputee, meaning that the socket axis 39 may not be centrally located.) Once in place, the DFMD 38 is mechanically coupled or confided within the socket. As a result, the location of the sensor measuring region 42 does not deviate from the original location while in use. In other words, regardless whether the patient is walking, running, sitting, wearing the prosthesis or any number of articles thereof, the sensor measuring region does not change location.

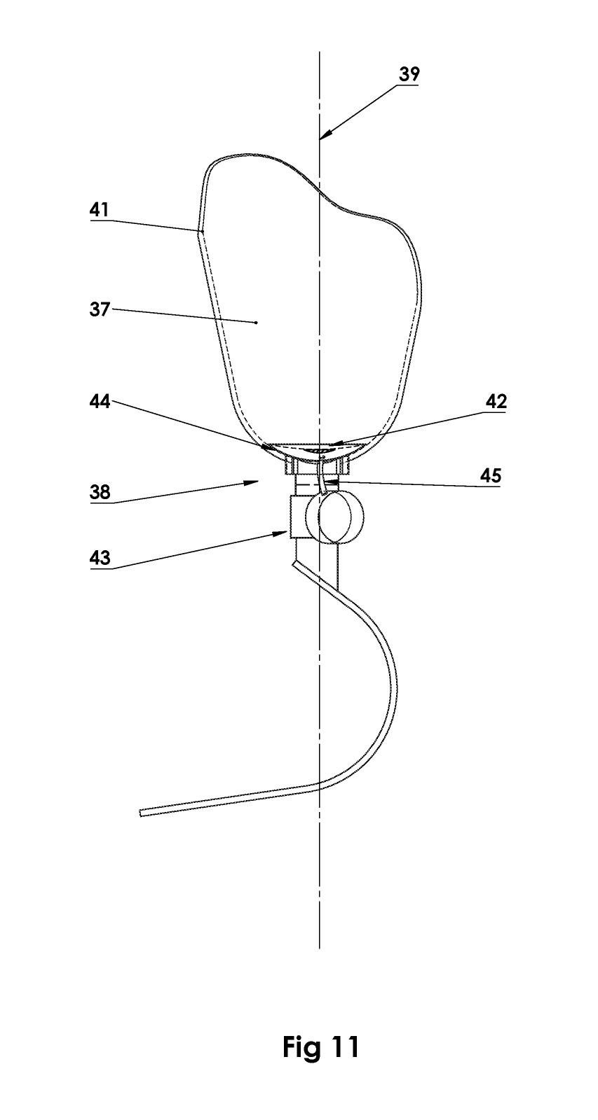

FIG. 11 shows an additional embodiment of the present invention in which, the DFMD 38 is split into a plurality of components. In a configuration of preferably two components, the first component, the processor enclosure 43, is external to the socket and is coupled to the prosthesis. The second component, the force sensor enclosure 44, is a remote unit attached to the processor by a multi-conductor cable 45. The sensor is affixed to the inside surface of the socket at the lowest point of the socket axis 39. In this embodiment, the processor enclosure contains the transceiver module, power supply (IE. battery), charging port, speaker, luminaries, and data ports (not shown). The force sensor housing is affixed to the inside surface of the socket 41 at the lowest point of the socket axis 39. As a result, the location of the sensor measuring region 42 does not deviate from the original location while in use.

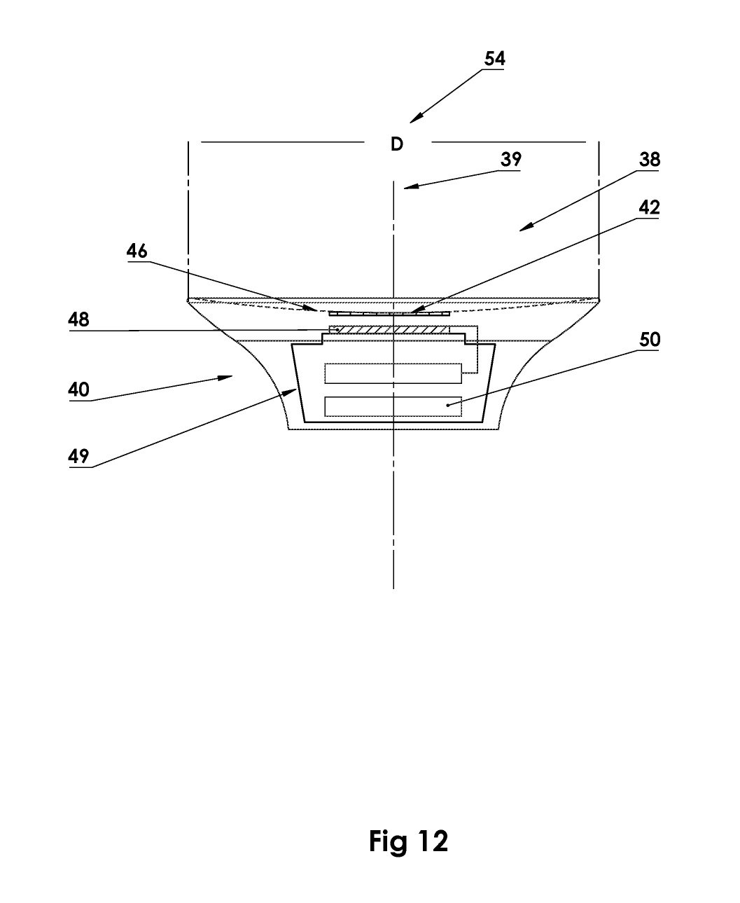

FIG. 12 illustrates a side section view of a first embodiment of the present invention. In this view, the DFMD 38 is a single enclosure 40 with concave upper surface 46 that is parallel with the distal end of the residual limb of the patient. The enclosure of the DFMD 38 in this embodiment has a coating 47 over at least the upper surface of the enclosure 40 that protects the force sensor from abrasion, water, corrosion that may occur during normal use of the prosthesis. The distal force applied by the residual limb must overcome the forces of the coating that are in the opposite direction of the distal forces of the residual limb. The resulting and opposite force of the coating would reduce the measurement of the force sensor if it were too rigid. The sensor measuring region 42 is defined as the surface on the upper concave surface of the DFMD that is directly above the sensor 48. Distal force is distributed along the upper surface of the concave surface which includes regions that are not within the sensor measuring region, or in other words, are not measured. As a result, the distal force measurement is relative to the forces only on the sensor measuring region. Due to the soft nature of the socks and liner the distal force is inherently distributed to some regions outside of the sensor measuring region that is not measured. The sensor is mounted to the top surface of the rigid circuitry enclosure 49. In this embodiment the enclosure houses the circuitry that is used to monitor sensor data and transmit said data wirelessly to an external device. The power supply 50 which is used to power the DFMD 38 is also stored within said enclosure.

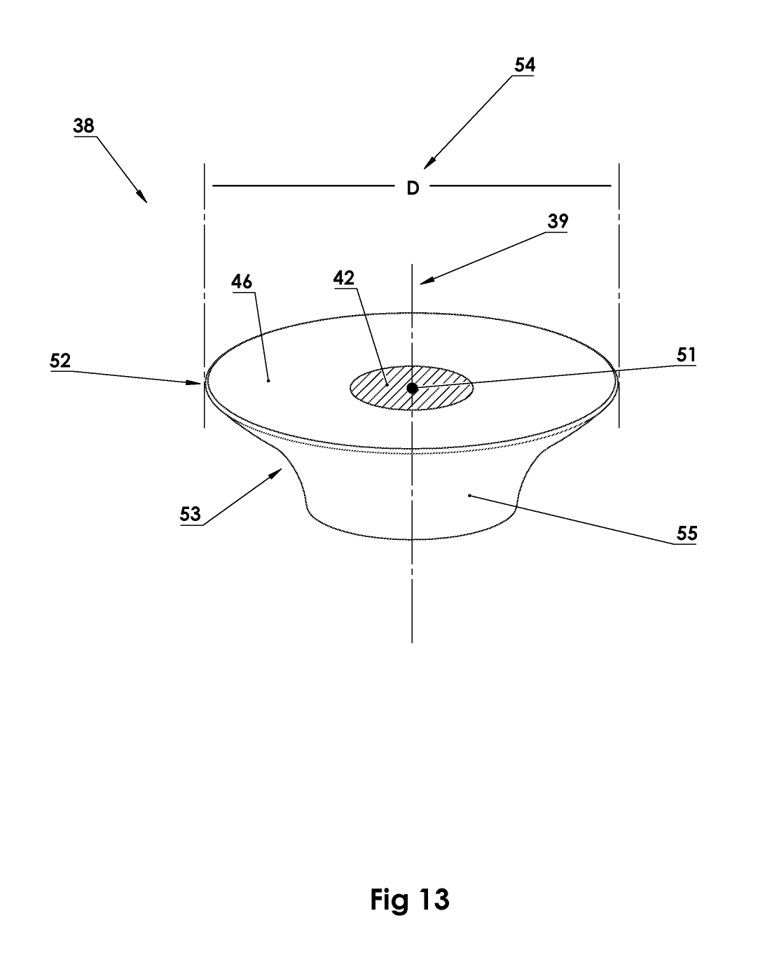

FIG. 13 shows an isometric view of the embodiment shown in FIG. 12. The sensor measuring region axis 39 preferably runs through the center point 51 of the DFMD sensor measuring region 42 and is collinear with the sensor measuring axis 39 of the socket, although the sensor measuring region axis 39 may be located at any point on the sensor. Preferably, the sensor measuring region 42 is concentric to the upper peripheral edge 52 of the DFMD 38. The sensor measuring region 42 is defined as the surface on the upper concave surface 46 of the DFMD 38 that is directly above the sensor. The upper concave surface 46 of the DFMD 38 is comprised of the upper surface sensor membrane 47. Preferably, the upper surface sensor membrane 47 is between 0.5 mm and 2 mm and is comprised of a polymer with a durometer greater than 70 Shore A. Alternatively, the upper surface sensor membrane 47 is between 3.0 and 25.0 mm and the coating is less than 65 Shore A. The upper surface sensor membrane 47 is configured to protect the sensor measuring region 42 from external wear factors and also to provide a uniform concave surface for the convex distal portion of the residual limb to mate to. The radius 54 of the upper concave surface 47 of the DFMD is between 50 and 600 percent of the greater diameter D 54 of the DFMD 38. The side surface 55 of the DFMD 38 is the surface below the greater peripheral edge of the DFMD and has a positive draft taper with respect to the area created by the upper peripheral edge of the DFMD.

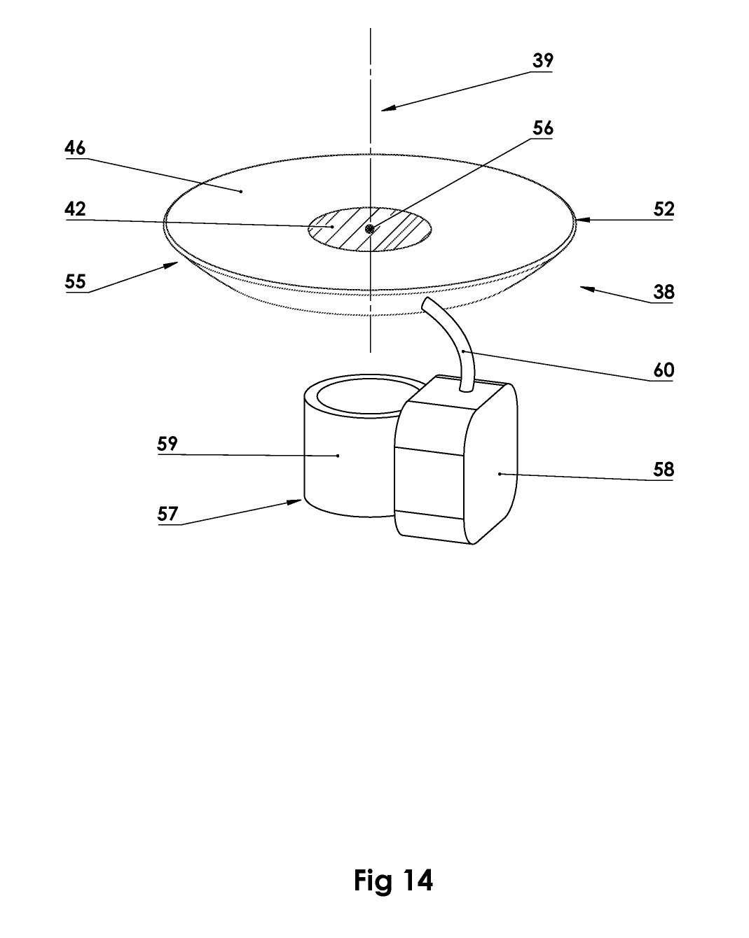

FIG. 14 illustrates a second embodiment of the present invention in which the DFMD 38 is comprised of two components. The first component, the sensor enclosure 55 is comprised of a sensor measuring axis 39 that preferably runs through the center point 56 of the DFMD sensor measuring region 42. Accordingly, the sensor measuring region 42 is preferably concentric to the upper peripheral edge 52 of the sensor enclosure and is located on the upper concave surface 46 directly above the sensor. The second component of the DFMD 38 in this embodiment is the processor enclosure 57. The processor enclosure is comprised of the circuitry enclosure 58 that houses the electrical components required to operate the device. The circuitry enclosure is comprised of a mechanical coupling 59 feature that allows for coupling of the processor enclosure to the prosthesis. In a preferred embodiment, the mechanical coupling is a 30 mm clamp that secures the processor enclosure to 30 mm tubing that is commonly to prosthesis construction. Alternatively, the mechanical coupling is a bracket, or plate that allows for mechanical fastening to the pylon, socket, or other feature of the prosthesis. In this embodiment the sensor enclosure is the only component that is affixed within the socket.

The processor enclosure and the sensor enclosure are coupled by a wire 60. The wire may be detachable from either the processor enclosure or the sensor enclosure via any commonly used detachable electronic connector. In this configuration the wire that connects the two enclosures is preferably routed through an aperture in the socket. Many alternatives exist such as routing the wire along the outside of the socket, over the upper peripheral socket edge, along the inside socket surface, and to the sensor enclosure.

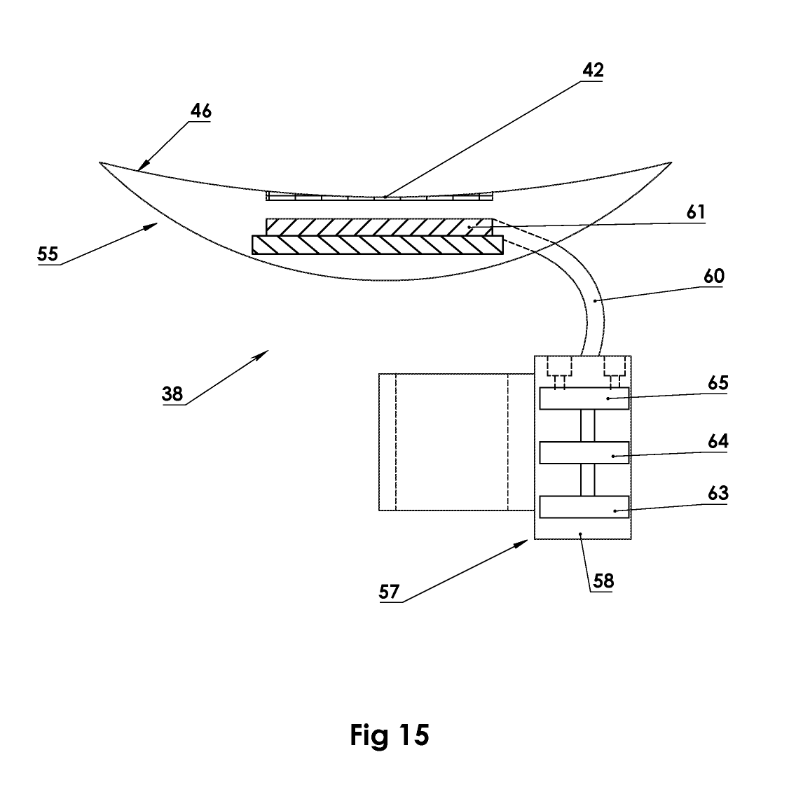

The illustration in FIG. 15 shows a side section view of the second embodiment shown in FIG. 14. This embodiment is comprised of a two component configuration in which the sensor enclosure 55 is located within the socket and the processor enclosure 57 outside of the socket coupled to the prosthesis. The sensor enclosure has a concave upper surface 46 wherein lies the sensor measuring region 42 on an axis collinear to the socket axis 39. Below the sensor measuring region 42 approximately 0.5 to 9 mm is the sensor 61. The sensor is affixed to the sensor mounting plate 62. The processor enclosure comprises the power supply 63, circuitry 64 related processing data, and a transceiver module 65 for transmittal of data to and from an external device. The transceiver module may utilize any common frequency reception or transmittal of data. The processor enclosure includes means for the attachment of data and/or power cables. These may include USB, micro-USB, Ethernet, or any similarly configured connection types.

The invention has been described in terms of various embodiments. It will be appreciated by those skilled in the art that various changes and modifications may be made to the embodiments without departing from the spirit or scope of the invention. It is not intended that the invention be limited to the embodiment shown and described. It is intended that the invention include all foreseeable modifications to the embodiments shown and described. It is intended that the invention be limited in scope only by the claims appended thereto.

What is claimed is:

1. A prosthesis assembly comprising:
a prosthetic socket having an open proximal end and a closed distal end, said socket having an tubular-shaped interior surface extending along a longitudinal axis thereof and configured to surround a residual limb of an amputated leg of an amputee when worn, said socket having a lower interior surface within said closed distal end;
a prosthetic liner comprising an interior surface configured to engage the residual limb when worn by the amputee and an exterior surface having a first section adapted to engage said upper tubular-shaped surface of said socket when worn by the amputee and a second section spaced from said interior surface of said closed distal end of said socket when worn by the amputee thereby defining a volume there between;
at least one force sensor affixed to a sensor mounting plate having a diameter mounted within said lower interior surface of said prosthetic socket aligned with said longitudinal axis of said prosthetic socket and in proximity to said lower interior surface of said prosthetic socket for measuring forces against said prosthetic socket created by a downward movement of the residual limb of said amputee when wearing said prosthesis and for electronically transmitting data indicative of said measured force;
a processor for electronically receiving said data from said at least one force sensor;
a transceiver module electrically communicating with said processor for wirelessly transmitting said data from said processor to a remote data processing computer; and
a power source electrically connected to said at least one force sensor, said processor and said transceiver module for powering the same.

2. The prosthesis assembly as claimed in claim 1, wherein said at least one force sensor, processor, transceiver module, and power source are removably mounted.

3. The prosthesis assembly of claim 1, wherein said at least one force sensor. processor, transceiver module, and power source are mechanically coupled.

4. The prosthesis assembly of claim 1, wherein said at least one force sensor is separated from said processor, transceiver module, and power source.

5. The prosthesis assembly of claim 1, wherein said at least one sensor comprises a concave upper surface, a force sensor having a sensor measuring region, a protective coating, and a circuitry enclosure.

6. The prosthesis assembly of claim 5, wherein the circuitry enclosure houses the processor, transceiver module, and power source.

7. The prosthesis assembly of claim 1, wherein said at least one sensor mounting plate comprises a force sensor, a concentric sensor enclosure comprising an upper peripheral edge, a concave upper surface, an upper surface sensor membrane, a sensor measuring central axis, a sensor measuring region collinear with the sensor measuring central axis, and a processing enclosure comprising a circuitry enclosure and coupler and connected to the sensor enclosure by a detachable wire.

8. The prosthesis assembly of claim 7, wherein said sensor measuring region is concentric to the upper peripheral edge.

9. The prosthesis assembly of claim 7, wherein said sensor measuring region is directly above the force sensor.

10. The prosthesis assembly of claim 7, wherein said circuitry enclosure houses the processor, transceiver module, and power source.

11. The prosthesis assembly of claim 7, wherein said coupler is a clamp.

12. The prosthesis assembly of claim 7, wherein said upper surface sensor membrane comprises silicone, polyurethane, copolymer, or block copolymer.

13. The prosthesis assembly of claim 12, wherein said upper surface sensor membrane is between 0.5 mm and 2.0 mm thick above the sensor.

14. The prosthesis assembly of claim 13, wherein said upper surface sensor membrane has a durometer greater than 70 Shore A.

15. The prosthesis assembly of claim 12, wherein said upper surface sensor membrane is between 3.0 mm and 25.0 mm thick above the sensor.

16. The prosthesis assembly of claim 15, wherein said upper surface sensor membrane has a durometer less than 65 Shore A.

17. The prosthesis assembly of claim 12, wherein said concave upper surface sensor membrane has a diameter between 50 and 600 percent of the sensor mounting plate diameter.

18. A prosthesis assembly comprising:
a prosthetic socket having an open proximal end and a closed distal end;
a prosthetic liner comprising an interior surface configured to engage a residual limb of an amputee and an exterior surface having a first section adapted to engage the prosthetic socket;
at least one force sensor affixed to a sensor mounting plate mounted within said lower interior surface of said prosthetic socket;
a processor for electronically receiving said data from said at least one force sensor;
a transceiver module electrically communicating with said processor for wirelessly transmitting said data from said processor to a remote data processing computer; and
a power source electrically connected to said at least one force sensor, said processor and said transceiver module for powering the same.

19. The prosthesis assembly of claim 18, wherein said prosthetic socket further comprises an upper tubular-shaped interior surface extending along a longitudinal axis thereof and configured to surround the residual limb an amputee when worn, said prosthetic socket having a lower interior surface within said closed distal end.

20. The prosthesis assembly of claim 19, wherein said at least one force sensor is offset from said longitudinal axis of said prosthetic socket and in proximity to said lower interior surface of said prosthetic socket for sensing forces against said prosthetic socket created by a downward movement of the residual limb of said amputee when wearing said prosthesis and for electronically transmitting data indicative of said measured force.

21. The prosthesis assembly of claim 18, wherein said at least one sensor, processor, transceiver module, and power source are removably mounted.

22. The prosthesis assembly of claim 18, wherein said at least one force sensor, processor, transceiver module, and power source are secured to each other.

23. The prosthesis assembly of claim 18, wherein said at least one force sensor is separated from said processor, transceiver module, and power source.

24. The prosthesis assembly of claim 18, wherein said at least one sensor comprises a concave upper surface, a force sensor having a sensor measuring region, a protective coating, and a circuitry enclosure.

25. The prosthesis assembly of claim 24, wherein the circuitry enclosure houses the processor, transceiver module, and power source.

26. The prosthesis assembly of claim 18, wherein said at least one sensor comprises a force sensor, a sensor enclosure comprising an upper peripheral edge, a concave upper surface, an upper surface sensor membrane, a sensor measuring central axis, a sensor measuring region collinear with the sensor measuring central axis, and a processing enclosure comprising a circuitry enclosure and coupler and connected to the sensor enclosure by a detachable wire.

27. The prosthesis assembly of claim 26, wherein said sensor measuring region is concentric to the upper peripheral edge.

28. The prosthesis assembly of claim 26, wherein said sensor measuring region is directly above the force sensor.

29. The prosthesis assembly of claim 26, wherein said circuitry enclosure houses the processor, transceiver module, and power source.

30. The prosthesis assembly of claim 26, wherein said coupler is a clamp.

31. The prosthesis assembly of claim 26, wherein said upper surface sensor membrane comprises silicone, polyurethane, copolymer, block copolymer, plastic, silicone, or fabric.

32. The prosthesis assembly of claim 31, wherein said upper surface sensor membrane is between 0.5 mm and 2.0 mm thick above the sensor.

33. The prosthesis assembly of claim 31, wherein said upper surface sensor membrane is between 3.0 mm and 25.0 mm thick above the sensor.

34. The prosthesis assembly of claim 31, wherein said upper surface sensor membrane has a diameter between 50 and 600 percent of the sensor mounting plate diameter.

35. A prosthesis assembly comprising:
a prosthetic socket having an open proximal end and a closed distal end, said socket having an tubular-shaped interior surface extending along an axis thereof and configured to surround a residual limb of an amputated leg of an amputee when worn, said socket having a lower interior surface within said closed distal end;
a prosthetic liner comprising an interior surface configured to engage the residual limb when worn by the amputee and an exterior surface having a first section adapted to engage said upper tubular-shaped surface of said socket when worn by the amputee and a second section spaced from said interior surface of said closed distal end of said socket when worn by the amputee thereby defining a volume therebetween;
at least one force sensor affixed to a sensor mounting plate having a diameter mounted within said lower interior surface of said prosthetic socket aligned with said axis of said prosthetic socket and in proximity to said lower interior surface of said prosthetic socket for sensing forces against said prosthetic socket created by a downward movement of the residual limb of said amputee when wearing said prosthesis and for electronically transmitting data indicative of said measured force;
a processor electronically communicating with said at least one force sensor for electronically receiving said data from said at least one force sensor;
a transceiver module electrically communicating with said processor for wireless electronically transmitting said data from said transceiver module to a remote data processing computer; and
a power source electrically connected to said at least one force sensor, said processor and said transceiver module for powering the same.

36. The prosthesis assembly of claim 35, wherein said axis is offset from a central point in the lower interior surface of said prosthetic socket.

37. The prosthesis assembly as claimed in claim 35, wherein said at least one force sensor, processor, transceiver module, and power source are removably mounted.

38. The prosthesis assembly of claim 35, wherein said at least one force sensor, processor, transceiver module, and power source are mechanically coupled.

39. The prosthesis assembly of claim 35, wherein said at least one force sensor is separated from said processor, transceiver module, and power source.

40. The prosthesis assembly of claim 35, wherein said at least one sensor comprises a concave upper surface, a force sensor having a sensor measuring region, a protective coating, and a circuitry enclosure.

41. The prosthesis assembly of claim 40, wherein the circuitry enclosure houses the processor, transceiver module, and power source.

42. The prosthesis assembly of claim 35, wherein said at least one sensor mounting plate comprises a force sensor, a concentric sensor enclosure comprising an upper peripheral edge, a concave upper surface, an upper surface sensor membrane, a sensor measuring axis, a sensor measuring region collinear with the sensor measuring axis, and a processing enclosure comprising a circuitry enclosure and coupler and connected to the sensor enclosure by a detachable wire.

43. The prosthesis assembly of claim 42, wherein said sensor measuring region is directly above the force sensor.

44. The prosthesis assembly of claim 42, wherein said circuitry enclosure houses the processor, transceiver module, and power source.

45. The prosthesis assembly of claim 42, wherein said coupler is a clamp.

46. The prosthesis assembly of claim 42, wherein said upper surface sensor membrane comprises silicone, polyurethane, copolymer, or block copolymer.

47. The prosthesis assembly of claim 46, wherein said upper surface sensor membrane is between 0.5 mm and 2.0 mm thick above the sensor.

48. The prosthesis assembly of claim 47, wherein said upper surface sensor membrane has a durometer greater than 70 Shore A.

49. The prosthesis assembly of claim 46, wherein said upper surface sensor membrane is between 3.0 mm and 25.0 mm thick above the sensor.

50. The prosthesis assembly of claim 49, wherein said upper surface sensor membrane has a durometer less than 65 Shore A.

51. The prosthesis assembly of claim 46, wherein said upper surface sensor membrane has a diameter between 50 and 600 percent of the sensor mounting plate diameter
